(19) United States
(12) Patent Application Publication
(10) Pub. No.: US 2026/0249289 A1
(43) Pub. Date: Aug. 27, 2026

(54) BIOCHIP, BIOLOGICAL SAMPLE TESTING DEVICE, AND A BIOLOGICAL SAMPLE TESTING METHOD

(71) Applicant: Radiant Opto-Electronics Corporation, KAOHSIUNG (TW)

(72) Inventors: Ya-Yin TSAI, KAOHSIUNG (TW); Hui-Hua HUNG, KAOHSIUNG (TW); Sung-Fu WU, KAOHSIUNG (TW); Yi-Jen CHIU, KAOHSIUNG (TW)

(73) Assignee: Radiant Opto-Electronics Corporation, KAOHSIUNG (TW)

(21) Appl. No.: 19/317,643

(22) Filed: Sep. 3, 2025

(30) Foreign Application Priority Data
Feb. 27, 2025 (CN) 202510223728.9

(51) Int. Cl. B01L 3/00

(52) U.S. Cl. CPC B01L 3/50273; B01L 3/502715; B01L 2200/027; B01L 2300/0816; B01L 2300/0819; B01L 2400/0406

(57) ABSTRACT

A biological chip comprises a chip unit and an elastic pad disposed on the chip unit. The chip unit includes an upper substrate and a lower substrate and is provided with a plurality of spaced-apart liquid inlets. A gap is formed between the upper and lower substrates for accommodating a test liquid. The liquid inlets are formed on the upper substrate and are in fluid communication with the gap. The elastic pad includes a plurality of injection ports and a plurality of suction ports, which are disposed therethrough in a spaced manner. The injection ports respectively correspond to the liquid inlets. The suction ports respectively correspond to the upper substrate and are configured to provide a suction force to the upper substrate so as to maintain the height of the gap, thereby preventing deformation of the biological chip and ensuring detection accuracy. The present invention further provides a biological sample testing device comprising the biological chip, and a method for analyzing a biological sample using the biological sample testing device.

### RELATED APPLICATIONS

This application claims priority to China Application Number 202510223728.9, filed on Feb. 27, 2025. The entire disclosures of all the above applications are hereby incorporated by reference.

### FIELD OF THE INVENTION

The present invention relates to a biochip, particularly a biochip capable of reducing deformation under applied force.

### BACKGROUND OF THE INVENTION

A biochip is a high-tech device designed based on principles of microelectronics, microfluidics, molecular biology, biotechnology, genetic testing, and analytical chemistry. It is fabricated using substrates such as silicon wafers, glass, or polymers, and manufactured through micro-electromechanical systems (MEMS) automation or other precision processing technologies. The biochip provides capabilities for biological testing and diagnostics that are rapid, accurate, and cost-effective.

Generally, a biochip must be used in conjunction with a liquid injection system. In conventional equipment, the liquid injection system operates by moving vertically, descending to abut with the biochip for liquid injection, and then ascending to separate from the biochip after the injection is completed. As shown in FIG. 10, a conventional biochip 90 comprises an upper substrate 91 and a lower substrate 92, with a gap G formed therebetween. However, when the liquid injection system descends and presses against the biochip, a downward pressure P1 is applied. During the process of injecting the test liquid through the injection port 93 into the gap G, the downward pressure P1 exerted by the injection system may cause deformation of the biochip, thereby reducing the gap G between the upper substrate 91 and the lower substrate 92. This deformation can also cause the liquid droplets injected by the system to become distorted, potentially resulting in overflow of the droplets into other microchannels or residual paths within the biochip, which may adversely affect the accuracy of the detection.

### SUMMARY OF THE INVENTION

One object of the present invention is to provide a biochip capable of overcoming deformation issues.

The biochip comprises a chip unit and an elastic pad disposed on the chip unit. The chip unit includes an upper substrate, a lower substrate, and a plurality of liquid inlets arranged at intervals, wherein a gap is formed between the upper substrate and the lower substrate for accommodating a detection liquid, and the liquid inlets are formed in the upper substrate and are in fluid communication with the gap. The elastic pad includes an upper surface and a lower surface, wherein the lower surface is attached to the chip unit, the elastic pad further includes a plurality of injection ports and a plurality of suction ports, which are spaced apart and extend through the upper surface and the lower surface of the elastic pad. Wherein, the injection ports respectively correspond to the liquid inlets of the chip unit, and the suction ports respectively correspond to the upper substrate of the chip unit, the suction ports being configured to provide a suction force to the upper substrate so as to maintain the height of the gap.

In a preferable embodiment, the chip unit further includes an adhesive layer disposed between the upper substrate and the lower substrate, the adhesive layer forming the gap between the upper substrate and the lower substrate, wherein the adhesive layer includes a generally annular surrounding portion and a plurality of grating portions laterally extending inward from the surrounding portion and arranged at intervals, each of the liquid inlets of the chip unit being located between two adjacent grating portions.

In a preferable embodiment, the chip unit further includes an adhesive layer disposed between the upper substrate and the lower substrate, and a rigid spacer layer disposed between the upper substrate and the lower substrate, the hardness of the rigid spacer layer is greater than that of the adhesive layer, the adhesive layer and the rigid spacer layer are arranged side by side, with the adhesive layer being positioned closer to the edge of the chip unit relative to the rigid spacer layer.

In a preferable embodiment, the chip unit further includes two adhesive layers disposed between the upper substrate and the lower substrate, and a rigid spacer layer disposed between the two adhesive layers, and the hardness of the rigid spacer layer is greater than that of the adhesive layers.

In a preferable embodiment, the elastic pad further includes a plurality of spaced-apart protruding docking portions, each of which corresponds to and is in fluid communication with the injection port and the suction port of the chip unit respectively.

Another object of the present invention is to provide a biological sample testing device, which comprises the aforementioned biochip and an operating apparatus. The operating apparatus comprises a liquid injection module and a suction module spaced apart from the liquid injection module. The liquid injection module corresponds to the injection ports of the biochip, and the suction module corresponds to the suction ports of the biochip, and the suction module provides a suction force to the upper substrate to maintain the gap height of the biochip.

In a preferable embodiment, the liquid injection module and the suction module are in planar contact with the elastic pad of the biochip.

In a preferable embodiment, the elastic pad further includes a plurality of spaced-apart protruding docking portions, the liquid injection module includes a plurality of downwardly extending injection connectors with gradually tapering outer diameters, the suction module includes a plurality of downwardly extending suction connectors with gradually tapering outer diameters, and the injection connectors and the suction connectors are respectively inserted into corresponding docking portions of the elastic pad.

Another object of the present invention is to provide a biological sample testing method, which comprises the operational steps of the aforementioned biological sample testing device.

The biological sample testing method sequentially includes an alignment step, a suction step, and an injection step. In the alignment step, the operating apparatus is brought close to the biochip until the operating apparatus comes into contact with the biochip and applies a downward pushing force to the biochip. In the suction step, the suction module of the operating apparatus provides an upward suction force to the upper substrate of the biochip via the suction port of the biochip to maintain the gap. In the injection step, the liquid injection module of the operating apparatus injects a liquid to be detected through the injection ports of the biochip.

In a preferable embodiment, the biological sample testing method further includes a termination step, wherein the termination step includes first stopping the injection step and then stopping the suction step.

In a preferable embodiment, prior to performing the alignment step, the gap of the biochip has a first height, and during the alignment step, due to the pushing force applied by the operating apparatus to the upper substrate of the biochip, the gap of the biochip has a second height, wherein the second height is less than the first height.

In a preferable embodiment, during the suction step, due to the suction force applied by the suction module to the upper substrate of the biochip, the gap of the biochip has a third height, wherein the third height is greater than the second height.

The characteristic of the biochip of the present invention is that during the injection process, the suction ports of the elastic pad can provide a suction force to the upper substrate of the chip unit, thereby maintaining the height of the gap of the chip unit. This prevents deformation of the biochip, thereby enhancing testing accuracy and improving testing efficiency.

### BRIEF DESCRIPTION OF THE DRAWINGS

FIG. 1 is a perspective view illustrating a first preferred embodiment of the biological chip according to the present invention.

FIG. 2 is a partial cross-sectional view illustrating the internal structure of the biological chip shown in FIG. 1.

FIG. 3 is a partial cross-sectional view illustrating a preferred embodiment of a biological sample detection device according to the present invention.

FIG. 4 is a flowchart illustrating a preferred embodiment of the biological sample detection method according to the present invention.

FIG. 5 is a partial side cross-sectional view illustrating another embodiment of an operating apparatus.

FIG. 6 is an exploded perspective view illustrating a second preferred embodiment of the biological chip according to the present invention.

FIG. 7 is a partial cross-sectional view illustrating the internal structure of the biological chip shown in FIG. 6.

FIG. 8 is a schematic view illustrating a third preferred embodiment of the biological chip according to the present invention.

FIG. 9 is a schematic view illustrating another variation of the third preferred embodiment.

FIG. 10 is a schematic view illustrating deformation of a liquid droplet of the test liquid during the injection process in a conventional biological chip.

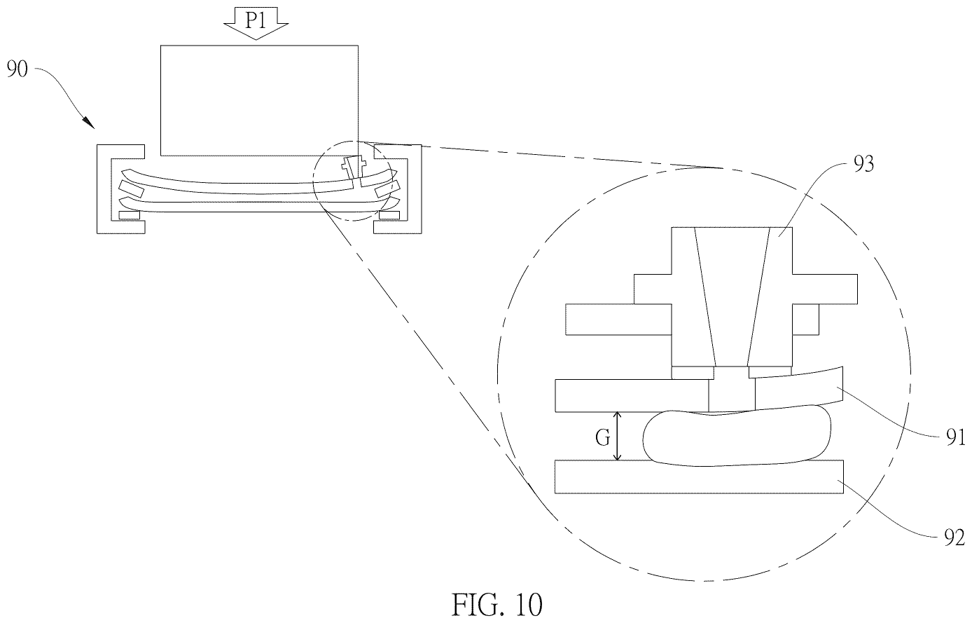

### DETAILED DESCRIPTION OF THE INVENTION

The detailed description and preferred embodiments of the invention will be set forth in the following content and provided for people skilled in the art to understand the characteristics of the invention. In addition, the directional terms mentioned in the following embodiments, such as up, down, left, right, front, back, bottom, top, etc., are only for reference to the directions in the attached drawings. Therefore, the directional terms used are illustrative rather than limiting.

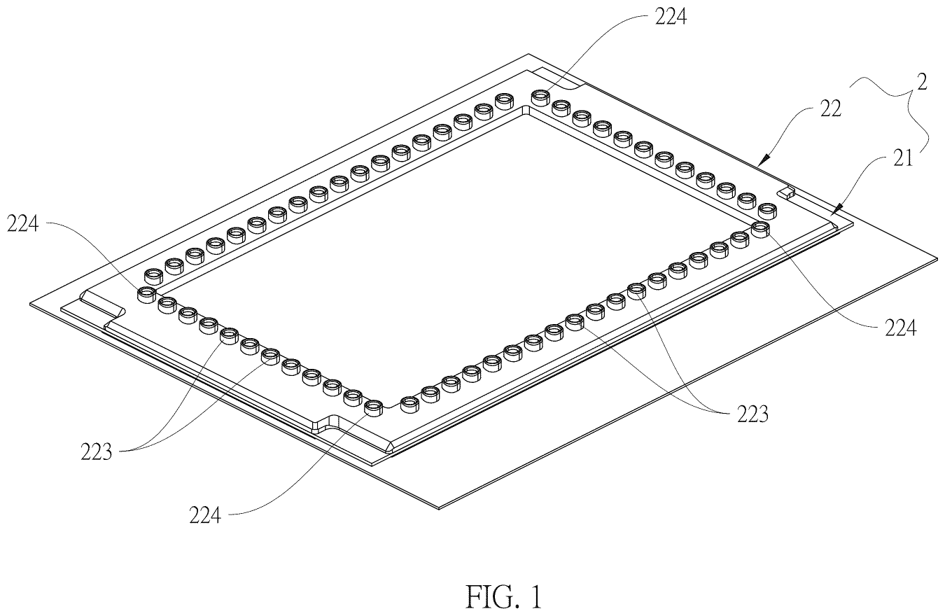

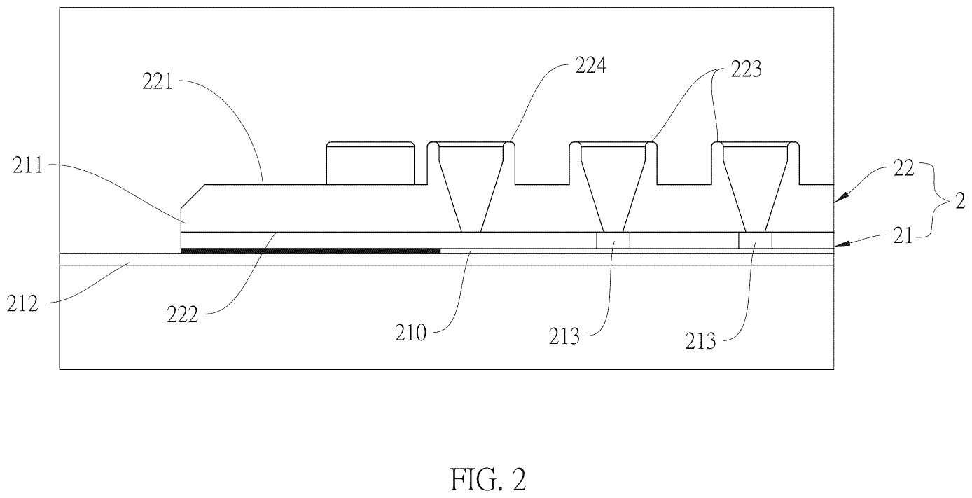

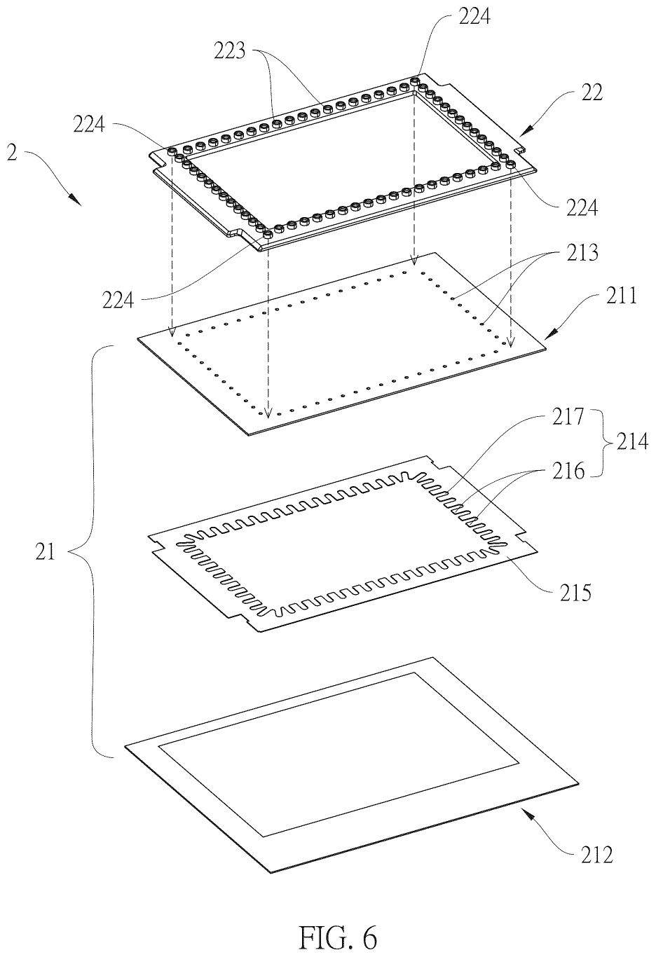

Referring to FIG. 1 and FIG. 2, it is a first preferred embodiment of the biological chip 2 according to the present invention. The biological chip 2 comprises a chip unit 21 and an elastic pad 22 disposed on the chip unit 21. As shown in FIG. 2, the chip unit 21 includes an upper substrate 211 and a lower substrate 212. The chip unit 21 is provided with a plurality of spaced-apart liquid inlets 213. A gap 210 is formed between the upper substrate 211 and the lower substrate 212 for accommodating a liquid to be tested. The liquid inlets 213 are formed in the upper substrate 211 and are in fluid communication with the gap 210. The elastic pad 22 has an upper surface 221 and a lower surface 222, wherein the lower surface 222 is attached to the chip unit 21. The elastic pad 22 includes a plurality of injection ports 223 and a plurality of suction ports 224, which are spaced apart and extend through both the upper surface 221 and the lower surface 222 of the elastic pad 22. Each of the injection ports 223 align with a respective liquid inlet 213 of the chip unit 21. Each of the suction ports 224 corresponds to the upper substrate 211 of the chip unit 21. More specifically, as long as the suction ports 224 correspond to regions of the upper substrate 211 where no liquid inlets 213 are formed, the suction ports 224 are able to adhere to the upper substrate 211 and provide a suction force thereto, thereby maintaining the height of the gap 210. Through the above-described structural design, during the liquid injection process, a suction force can be applied to the upper substrate 211 of the chip unit 21 via the suction ports 224 of the elastic pad 22. This prevents deformation of the chip unit 21 caused by downward pressure during injection. As a result, the height of the gap 210 between the upper substrate 211 and the lower substrate 212 of the chip unit 21 can be maintained, thereby preventing deformation of the biological chip 2 and improving detection accuracy and detection efficiency. For example, in the present embodiment shown in FIG. 1, the injection ports 223 of the biological chip 2 are arranged along the four sides, and at least one suction port 224 is provided on each side. In other embodiments, as illustrated in FIG. 6, the suction ports 224 may be positioned at the corners between the four sides.

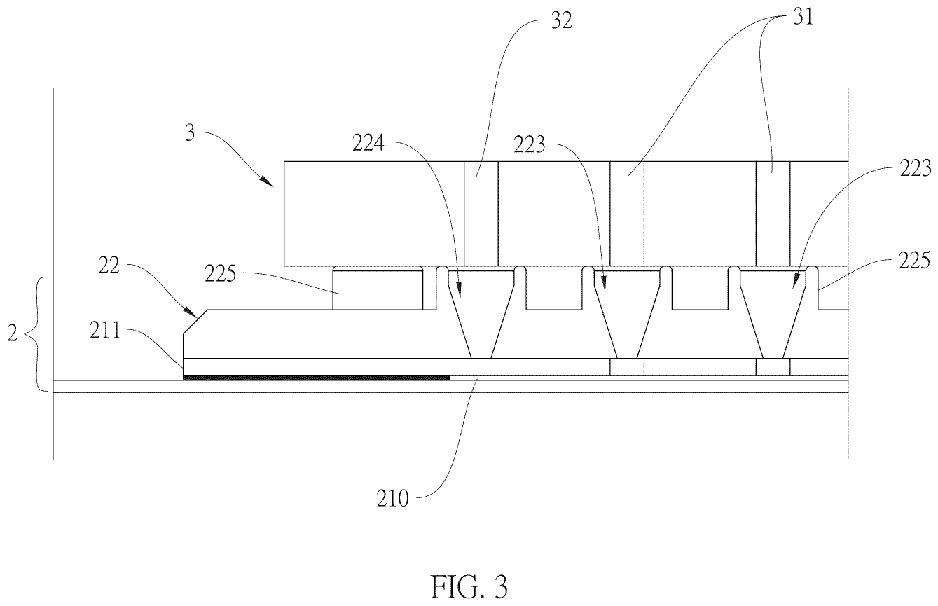

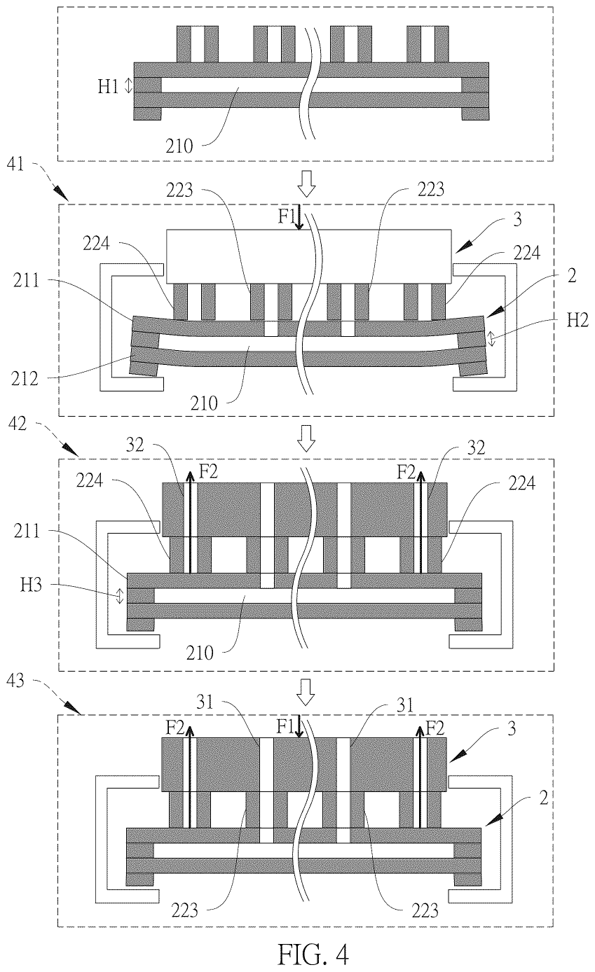

Referring to FIG. 3, it is a preferred embodiment of the biological sample testing device according to the present invention. The device includes the biochip 2 and an operating apparatus 3. The operating apparatus 3 comprises a liquid injection module 31 and a suction module 32 spaced apart from the liquid injection module 31. The liquid injection module 31 is aligned with the injection ports 223 of the biochip 2, and the suction module 32 corresponds to the suction ports 224 of the biochip 2. The suction module 32 provides a suction force to the upper substrate 211 to maintain the height of the gap 210 in the biochip 2. For example, the biological sample testing device of the present invention is applied in systems utilizing electromagnetic techniques to control the movement and mixing of the test liquid and reagents. Therefore, maintaining the height of the gap 210 between the upper substrate 211 and the lower substrate 212 of the biochip 2 not only prevents the injected test liquid and reagents from accumulating near the injection ports and failing to properly flow into the chip's operation zone, but also avoids unintended mixing of the liquids outside the operation zone. Accordingly, the biological sample testing device of the present invention contributes to improved detection accuracy and efficiency. The following describes a biological sample testing method of the present invention, implemented using the detection device shown in FIG. 3. Referring to FIG. 4, the testing method sequentially includes an alignment step 41, a suction step 42, and an injection step 43. It should be noted that, in FIG. 4, the structures of the biochip 2 and the operating apparatus 3 are illustrated schematically. The alignment step 41 involves bringing the operating apparatus 3 and the biochip 2 into proximity until the operating apparatus 3 comes into contact with the biochip 2, and a downward pushing force F1 is applied by the operating apparatus 3 onto the biochip 2. The suction step 42 involves the suction module 32 of the operating apparatus 3 applying an upward suction force F2 to the upper substrate 211 of the biochip 2 through the suction ports 224, in order to maintain the gap 210. The injection step 43 involves the liquid injection module 31 of the operating apparatus 3 injecting a test liquid through the injection ports 223 of the biochip 2. When the operating apparatus 3 comes into contact with the biochip 2, a downward pushing force F1 is applied to the upper substrate 211 of the biochip 2. This may cause deformation of the upper substrate 211, resulting in a downward deflection (as shown in the alignment step 41), which narrows the gap 210 between the upper substrate 211 and the lower substrate 212. As a result, the injected test liquid may have difficulty moving according to the intended instructions. Therefore, when the liquid injection module 31 comes into contact with the biochip 2 and applies the downward pushing force F1 to the upper substrate 211, the upward suction force F2 provided by the suction module 32 helps reduce the amount of deformation of the upper substrate 211, thereby maintaining the gap 210 without narrowing.

More specifically, before executing the alignment step 41, the gap 210 of the biochip 2 has a first height H1. During the execution of the alignment step 41, due to the pushing force F1 applied by the operating apparatus 3 to the upper substrate 211 of the biochip 2, the gap 210 is reduced to a second height H2, where the second height H2 is less than the first height H1. During the execution of the suction step 42, the suction force F2 applied by the suction module 32 to the upper substrate 211 of the biochip 2 causes the gap 210 to expand to a third height H3, where the third height H3 is greater than the second height H2. In other words, during the execution of the alignment step 41, the pushing force F1 applied by the operating apparatus 3 to the upper substrate 211 of the biochip 2 presses downward on the biochip 2, thereby reducing the gap 210 (the second height H2 is smaller than the first height H1). During the execution of the suction step 42, the suction force F2 applied by the suction module 32 pulls the upper substrate 211 upward, increasing the distance between the upper substrate 211 and the lower substrate 212. As a result, the previously compressed gap of the biochip 2 is enlarged again, such that the third height H3 is greater than the second height H2. Through this mechanism, the gap 210 of the biochip 2 can be maintained without narrowing during the liquid injection process, thereby allowing the injected test liquid droplets to retain their shape and move smoothly.

The biological sample testing method further includes a termination step. In this step, the injection step 43 is stopped first, followed by the suction step 42. This ensures that the liquid injection process is completed before the suction force applied to the biochip 2 is released, thereby maintaining the height of the gap 210 in the biochip 2 throughout the injection process.

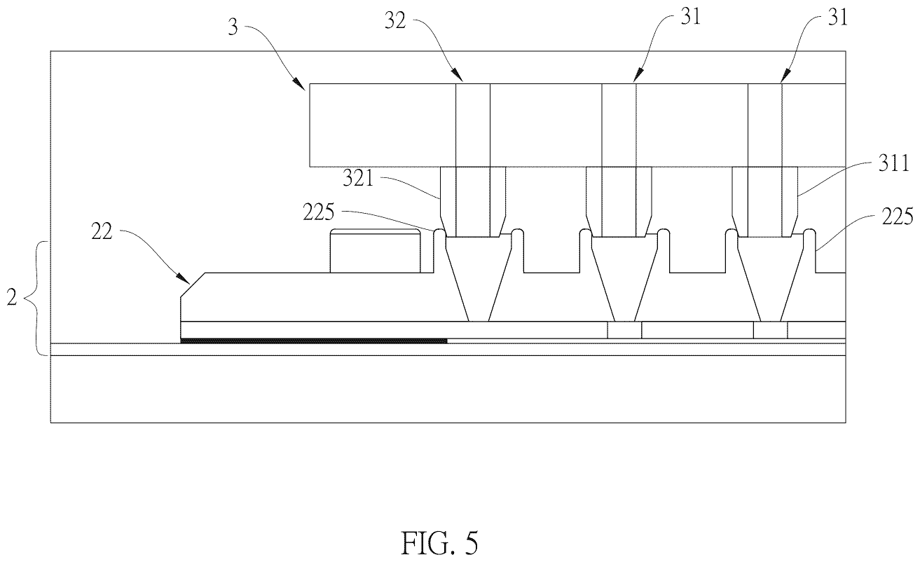

Referring to FIG. 3, the elastic pad 22 further includes a plurality of spaced-apart protruding docking portions 225. The docking portions 225 respectively correspond to and are in fluid communication with the injection ports 223 and the suction ports 224 of the chip unit 21. In the present embodiment, both the liquid injection module 31 and the suction module 32 are in planar contact with the elastic pad 22 of the biochip 2. In other embodiments, as shown in FIG. 5, the liquid injection module 31 includes a plurality of downwardly extending injection connectors 311, each having a gradually tapered outer diameter. Similarly, the suction module 32 includes a plurality of downwardly extending suction connectors 321, each also having a gradually tapered outer diameter. The injection connectors 311 and suction connectors 321 are inserted into the corresponding docking portions 225 of the elastic pad 22. With this structural design, if any misalignment occurs during the approach of the operating apparatus 3 toward the biochip 2 (as illustrated in FIG. 5), the tapered shapes of the injection connectors 311 and the suction connectors 321 serve a guiding function. This ensures that the connectors are properly aligned and inserted into the corresponding docking portions 225 of the elastic pad 22, thereby improving alignment accuracy.

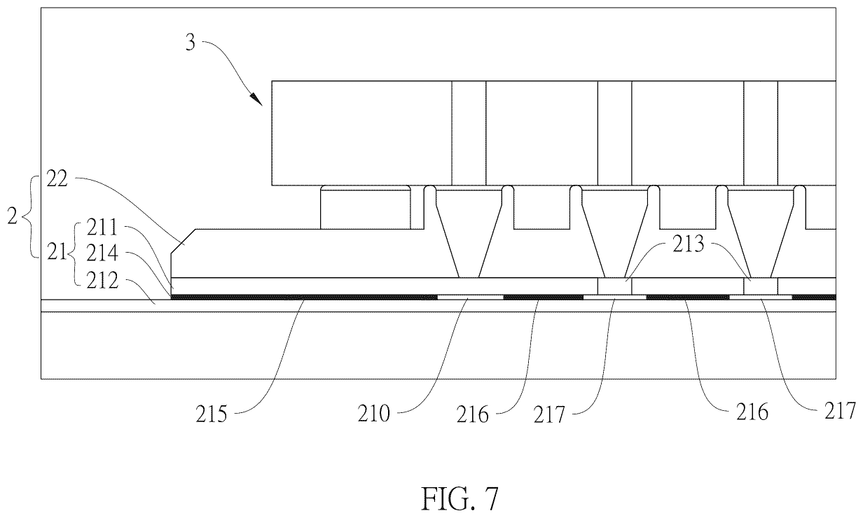

Referring to FIG. 6 and FIG. 7, it is a second preferred embodiment of the biochip 2 according to the present invention. The biochip 2 includes a chip unit 21 and an elastic pad 22 disposed on the chip unit 21. The chip unit 21 comprises an upper substrate 211, a lower substrate 212, and an adhesive layer 214 disposed between the upper substrate 211 and the lower substrate 212. The adhesive layer 214 defines a gap 210 between the upper substrate 211 and the lower substrate 212. In the present embodiment, the injection ports 223 are arranged along the four sides of the elastic pad 22, and one suction port 224 is provided at each of the four corners. As shown in FIG. 6, the adhesive layer 214 includes an annular surrounding portion 215 and a plurality of grating portions 216 extending laterally inward from the surrounding portion 215 in a spaced arrangement. Through the design of the adhesive layer 214 with the annular surrounding portion 215 and the grating portions 216, uniform support is provided along the periphery between the upper substrate 211 and the lower substrate 212, thereby ensuring the uniformity of the gap 210 throughout the entire biochip 2. The chip unit 21 has a plurality of injection inlets 213 arranged at intervals. The injection inlets 213 are formed in the upper substrate 211 and are in communication with the gap 210. Each of the injection inlets 213 is located between two adjacent grating portions 216 of the adhesive layer 214. More specifically, a channel 217 is formed between each pair of adjacent grating portions 216, and each channel 217 corresponds to one injection inlet 213. The biochip 2 of this preferred embodiment can also cooperate with the operating apparatus 3. After the test liquid is injected through the injection inlets 213, it is confined within the channels 217 formed between adjacent grating portions 216 and flows toward the operating region of the chip unit 21. From the above, it can be understood that the adhesive layer 214 not only serves to maintain the gap 210, but also, through the grating portions 216, effectively isolates adjacent injection inlets 213. This prevents cross-over or overflow of test liquids between different injection inlets 213, thereby ensuring the accuracy of the biological reaction detection. The grating portions 216 also reduce the cavity space located beneath each injection inlet 213 of the chip unit 21, ensuring that the test liquid, once injected, can smoothly flow toward the operating region of the chip unit 21. This design prevents residual fluid accumulation beneath the injection inlets 213 and simultaneously contributes to an increase in the effective detection volume.

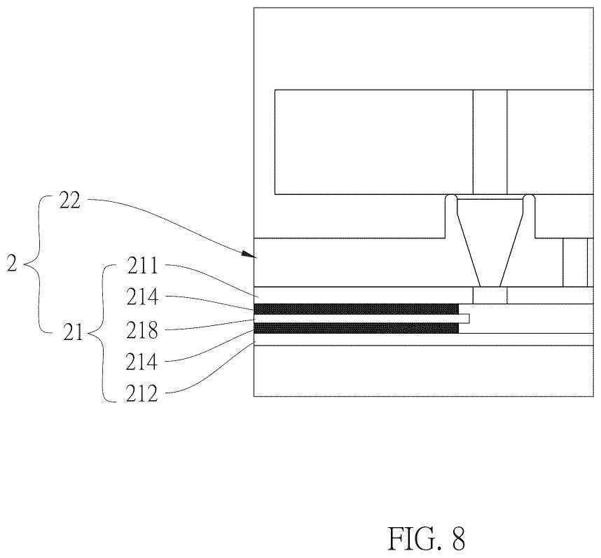

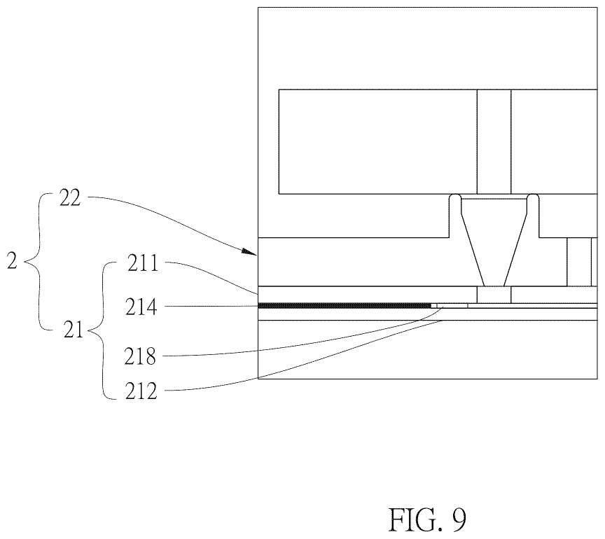

Referring to FIG. 8, a third preferred embodiment of the biochip 2 of the present invention is illustrated. Features identical to those of the first preferred embodiment will not be described again. The difference lies in that the chip unit 21 further includes two adhesive layers 214 disposed between the upper substrate 211 and the lower substrate 212, and a rigid spacer layer 218 positioned between the two adhesive layers 214. The hardness of the rigid spacer layer 218 is greater than that of the adhesive layers 214. By incorporating the rigid spacer layer 218, the overall rigidity of the chip unit 21 can be effectively enhanced, thereby preventing deformation of the chip unit 21 during the liquid injection process due to external forces. Referring to FIG. 9, in other embodiments, the configuration of the adhesive layer 214 and the rigid spacer layer 218 may alternatively be such that one adhesive layer 214 and one rigid spacer layer 218 are arranged side by side, with the adhesive layer 214 positioned closer to the edge of the chip unit 21 than the rigid spacer layer 218. In this way, not only is the overall rigidity of the biochip 2 improved, but the thickness of the biochip 2 can also be further reduced. It should be noted that in FIG. 8 and FIG. 9, the thicknesses of the adhesive layers 214 and the rigid spacer layer 218 are for illustrative purposes only and may be adjusted according to actual requirements.

In summary, the biochip 2 of the present invention provides the characteristics that, during the liquid injection process, a suction force is applied to the upper substrate 211 of the chip unit 21 via the suction port 224 of the elastic pad 22. This suction force helps maintain the height of the gap 210 within the chip unit 21, thereby preventing deformation of the biochip 2 and avoiding liquid overflow that could affect detection accuracy. Furthermore, through the incorporation of the rigid spacer layer 218, the overall rigidity of the biochip 2 is further enhanced, thereby reducing the likelihood of deformation during operation. In addition, with the advancement of biomedical technologies and the corresponding expansion of the market, the application scope of the biochip 2 of the present invention has been increasingly broadened. As the number of liquid samples required for detection within the chip module continues to grow, the biochip 2 can be more extensively applied in fields such as biological sample testing, including high-throughput screening, disease diagnosis, drug development, clinical diagnostics, and environmental monitoring. Compared to conventional chip modules, the modifications made to the internal structure of the biological sample testing device and the structural design of the fixtures within the chip module not only resolve existing issues and enhance operational efficiency but also achieve these improvements without the need for additional mechanical components. By altering only certain functions and structural features, the present invention reduces design costs, improves usability, and increases overall return on investment.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention covers modifications and variations of this invention provided they fall within the scope of the following claims.

What is claimed is:

1. A biochip, comprising:
a chip unit, including an upper substrate, a lower substrate, and a plurality of liquid inlets arranged at intervals, wherein a gap is formed between the upper substrate and the lower substrate for accommodating a detection liquid, and the liquid inlets are formed in the upper substrate and are in fluid communication with the gap; and
an elastic pad, including an upper surface and a lower surface, wherein the lower surface is attached to the chip unit, the elastic pad further includes a plurality of injection ports and a plurality of suction ports, which are spaced apart and extend through the upper surface and the lower surface of the elastic pad;
wherein the injection ports respectively correspond to the liquid inlets of the chip unit, and the suction ports respectively correspond to the upper substrate of the chip unit, the suction ports being configured to provide a suction force to the upper substrate so as to maintain the height of the gap.

2. The biochip as claimed in claim 1, wherein the chip unit further includes an adhesive layer disposed between the upper substrate and the lower substrate, the adhesive layer forming the gap between the upper substrate and the lower substrate, wherein the adhesive layer includes a generally annular surrounding portion and a plurality of grating portions laterally extending inward from the surrounding portion and arranged at intervals, each of the liquid inlets of the chip unit being located between two adjacent grating portions.

3. The biochip as claimed in claim 1, wherein the chip unit further includes an adhesive layer disposed between the upper substrate and the lower substrate, and a rigid spacer layer disposed between the upper substrate and the lower substrate, the hardness of the rigid spacer layer is greater than that of the adhesive layer, the adhesive layer and the rigid spacer layer are arranged side by side, with the adhesive layer being positioned closer to the edge of the chip unit relative to the rigid spacer layer.

4. The biochip as claimed in claim 1, wherein the chip unit further includes two adhesive layers disposed between the upper substrate and the lower substrate, and a rigid spacer layer disposed between the two adhesive layers, and the hardness of the rigid spacer layer is greater than that of the adhesive layers.

5. The biochip as claimed in claim 1, wherein the elastic pad further includes a plurality of spaced-apart protruding docking portions, each of which corresponds to and is in fluid communication with the injection port and the suction port of the chip unit respectively.

6. A biological sample testing device, comprising:
the biochip as described in claim 1; and
an operating apparatus, comprising a liquid injection module and a suction module spaced apart from the liquid injection module, the liquid injection module corresponds to the injection ports of the biochip, and the suction module corresponds to the suction ports of the biochip, and the suction module provides a suction force to the upper substrate to maintain the gap height of the biochip.

7. The biological sample testing device as claimed in claim 6, wherein the liquid injection module and the suction module are in planar contact with the elastic pad of the biochip.

8. The biological sample testing device as claimed in claim 6, wherein the elastic pad further includes a plurality of spaced-apart protruding docking portions, the liquid injection module includes a plurality of downwardly extending injection connectors with gradually tapering outer diameters, the suction module includes a plurality of downwardly extending suction connectors with gradually tapering outer diameters, and the injection connectors and the suction connectors are respectively inserted into corresponding docking portions of the elastic pad.

9. A biological sample testing method, comprising operational steps for the biological sample testing device as described in claim 6, sequentially including:
an alignment step, wherein the operating apparatus is brought close to the biochip until the operating apparatus comes into contact with the biochip and applies a downward pushing force to the biochip;
a suction step, wherein the suction module of the operating apparatus provides an upward suction force to the upper substrate of the biochip via the suction port of the biochip to maintain the gap; and
an injection step, wherein the liquid injection module of the operating apparatus injects a liquid to be detected through the injection ports of the biochip.

10. The biological sample testing method as claimed in claim 9, further includes a termination step, wherein the termination step includes first stopping the injection step and then stopping the suction step.

11. The biological sample testing method as claimed in claim 9, wherein, prior to performing the alignment step, the gap of the biochip has a first height, and during the alignment step, due to the pushing force applied by the operating apparatus to the upper substrate of the biochip, the gap of the biochip has a second height, wherein the second height is less than the first height.

12. The biological sample testing method as claimed in claim 11, wherein during the suction step, due to the suction force applied by the suction module to the upper substrate of the biochip, the gap of the biochip has a third height, wherein the third height is greater than the second height.
